(19) United States
(12) Patent Application Publication
(10) Pub. No.: US 2024/0423828 A1
(43) Pub. Date: Dec. 26, 2024

(54) DILATION STENT FOR GENIOGLOSSUS MUSCLE FOR IMPROVING RESPIRATORY AIRWAY DURING HYPOPNEA

(71) Applicant: GUANGDONG JIUCCO MEDICAL EQUIPMENT CO., LTD., Guangdong (CN)

(72) Inventors: Hua LU, Guangdong (CN); Jiejing LU, Guangdong (CN)

(73) Assignee: GUANGDONG JIUCCO MEDICAL EQUIPMENT CO., LTD., Guangdong (CN)

(21) Appl. No.: 18/710,197

(22) PCT Filed: Nov. 11, 2022
(86) PCT No.: PCT/CN2022/131444

§ 371 Date: May 15, 2024

(30) Foreign Application Priority Data
Nov. 15, 2021 (CN) 202111344551.6

(51) Int. Cl. A61F 5/56; A61M 29/00

(52) U.S. Cl. CPC A61F 5/566; A61M 29/00; A61M 2210/0643

(57) ABSTRACT

A dilation stent is used to support the genioglossus muscle, and includes a sleeve, a central tube and a stent body, the central tube passes through and is sleeved in the sleeve, and the stent body is connected to an end of the sleeve; and several support arms are provided between the sleeve and the stent body, and the support arms are of a collapsible structure, with one end of each support arm being connected to the sleeve and the central tube, and the other end thereof being connected to the stent body. When the stent contracts, the support arms are collapsed, and the stent body is retracted at the end of the sleeve; and when the stent expands, the sleeve pushes the support arms to splay out, and thus the stent body spreads out.

### TECHNICAL FIELD

The present invention relates to the technical field of medical auxiliary devices, and in particular to, a genioglossus-dilating stent for improving a respiratory airway to reduce hypopnea.

### BACKGROUND

Difficulty breathing, a common symptom, occurs in the middle stage of novel coronavirus, frequently causing a sensation of oxygen deficiency and an inability to breathe normally. As a result, a ventilator or other auxiliary equipment need to be used to facilitate ventilation. Furthermore, breathing difficulties can be also caused by obstructive sleep apnea syndrome. When a patient having such syndrome sleeps in a supine position, with the body muscles relaxed, the genioglossus at the root of the tongue sags under the influence of gravity. This can obstruct the airway and lead to breathing pauses. Its high-risk group includes individuals who are obese or elderly with muscle relaxed, or who have narrowed airway structures, enlarged tonsils, short lower jaws, or developed airway swelling due to long-term smoking. Severe cases may experience throat blockage during sleep, making it difficult to breathe and posing a risk of suffocation. Currently, the treatment for obstructive sleep apnea syndrome is typically carried out through surgical removal of the tonsils. During such traumatic surgery, however, complications cause some potential risks, and thus this surgery may not be suitable for all patients. Therefore, external assistive devices are commonly used to support treatment as well.

In the related art, there is a device used to prevent breathing pauses. This device is inserted into the mouth, causing the left and right cheeks at the base of the mouth to puff outward. Under the action of the muscles connected to the cheeks being pulled outward, the extrinsic tongue muscles, including the genioglossus, hyoglossus muscle, and palatoglossus muscle, are pulled in the left and right directions. As a result, even when the patient is in a supine position during sleep, the genioglossus, hyoglossus muscle, and palatoglossus muscle, although they are relaxed, are difficult to extend in the direction of obstructing the airway, that is, a direction towards the back. Thereby, the root of the tongue has difficulty sagging towards the back, preventing it from obstructing the pharyngeal airway. However, because of the relatively large structural volume of the device, after inserted into the mouth, it causes discomfort with a strong sensation of foreign body to the patient, thus affecting the quality of the patient's sleep.

A recyclable heart stent has been disclosed in patent application No. CN 107811727A, which employs an elastic expandable mesh bag inserted in the vessels to provide support. However, the use of this heart stent requires an additional catheter for insertion and retrieval, and a heating apparatus for cooperation, making it unsuitable for direct application in respiratory airway dilation.

### SUMMARY

To help treat obstructive sleep apnea syndrome, the present invention provides a genioglossus-dilating stent for improving a respiratory airway to reduce hypopnea. The dilating stent, with a relatively small volume, can enter the human respiratory airway through the mouth or the nasal cavity, and are flexibly expanded therein, to support the genioglossus at the root of the tongue and prevent it from sagging and obstructing the airway when the patient is lying flat, thus avoiding respiratory pauses.

To resolve the foregoing technical problem, the present invention uses the following technical solution:

A genioglossus-dilating stent for improving a respiratory airway to reduce hypopnea is used to support human genioglossus, including a sleeve tube, a central tube, and a stent body. The central tube penetrates through and is sleeved within the sleeve tube, and the stent body is connected to an end portion of the sleeve tube; multiple support arms are provided between the sleeve tube and the stent body, the support arm is of a foldable structure, and the support arm has one end connected to the sleeve tube and the central tube and the other end connected to the stent body. During retraction, the support arms are folded, and the stent body is retracted at the end portion of the sleeve tube; and during expansion, the sleeve tube pushes the support arms to open, so as to expand the stent body.

During retraction, the entire dilating stent is in a shape of a slender and flexible tube. During use, one end of the stent body is inserted from the human nasal cavity or mouth to the position of the genioglossus in the airway. Subsequently, the stent body is expanded to prevent the genioglossus from sagging to obstruct breathing when the patient is lying flat. During the operation of the dilating stent, the central tube and the sleeve tube slide relative to each other, driving the support arms to open or close, thus expanding or retracting the stent body.

In addition, the central tube is longer than the sleeve tube, and the two are in clearance fit; and one end of the central tube away from the stent body is provided with a hook. When the dilating stent is in use, one end of the stent body is inserted into the human respiratory airway, while the other end remains outside the body. The stent body is expanded and retracted by manipulating the central tube and sleeve tube located outside the body. During use, to prevent the central tube and sleeve tube from extending too far into the nasal cavity, the hook at the rear end of the central tube is hooked onto the nose. This ensures greater safety during use and makes it suitable for both medical and home use.

As a preferred solution, the support arm is provided in a quantity of two, and the two support arms are symmetrically arranged around a central axis of the sleeve tube. To ensure ventilation, two or three support arms are provided. Too dense distribution of the support arms is not conducive to air circulation, impacting breathing quality.

As a preferred solution, the stent body is of an elastic mesh structure, and the stent body is pushed by the support arms to expand or retract. In general, the elastic mesh may be of a rhombus-shaped structure, a regular polygon-shaped structure, or a spiral structure. The structure itself possesses a certain level of strength and flexibility, and thus is retractable and expandable.

As a preferred solution, the stent body is of a toroidal structure, and the stent body is co-axially arranged with the sleeve tube; and the support arm is connected to an inner wall of the stent body. The stent body may be a complete toroidal structure, and the support arms pull the stent body to radially expand or retract.

As a preferred solution, the stent body is of an arc-shaped structure with a central angle not greater than 180°, the stent body and the support arm are provided in a same quantity, and during retraction, the stent bodies gather to be in a toroidal shape; and the support arms are connected to the stent bodies in one-to-one correspondence. The stent body may further be of an independent arc-shaped structure, and individual arc-shaped stent bodies are combined to be in a toroidal shape. Generally, one support arm is connected to one arc-shaped stent body, the support arm is connected to the inner wall of the stent body, and the arc-shaped stent body is symmetrically designed.

As a preferred solution, an outer diameter of expansion of the stent body is 15 mm to 25 mm. On the basis of the measurement requirements of ventilation amount, it is proper to ensure that the width of the respiratory airway is approximately 20 mm, with a maximum not exceeding 25 mm. Excessively large expansion is likely to cause damage to the pharyngeal mucosa and capillaries, while excessively small expansion does not achieve ventilation. Stent body

As a preferred solution, the support arm includes a support rod and a connection rod, and the support rod has one end hinged to the sleeve tube and the other end hinged to the inner wall of the stent body; and the connection rod has one end hinged to an end portion of the central tube and the other end hinged to a middle part of the support rod. The central tube penetrates through the sleeve tube, and therefore, the connection rod has one end hinged to the end portion extending from the central tube and the other end connected to the middle part of the support rod, so as to be in linkage with the support rod. In addition, the support rod has one end hinged to the sleeve tube and the other end hinged to the inner wall of the stent body. The support rod pulls the stent body to expand or retract.

As a preferred solution, the sleeve tube and the central tube are both made of a bendable and flexible material. Generally, a well-guiding non-toxic, harmless, and flexible material is selected.

As a preferred solution, one end of the central tube away from the stent body is provided with an elastic limit block, the sleeve tube is provided with multiple limit holes in fit with the elastic limit block, and the limit holes are spaced apart linearly and uniformly. During expansion, the elastic limit block is stuck in one of the limit holes, keeping the central tube relatively fixed to the sleeve tube. After expansion through support, to prevent the stent body from being retracted due to the pressure of the throat muscles, it is necessary to maintain the support force of the support arms on the stent body. Therefore, it is essential to keep the relative position between the central tube and sleeve tube fixed without undesired sliding. With multiple different limit holes provided to limit positions, when the positions of the central tube and the sleeve tube are limited through the cooperation between the elastic limit blocks with different limit holes, the stent is expanded to different levels and effectively fixed.

Compared with the related art, the present invention has the following beneficial effects: The present invention discloses a genioglossus-dilating stent for improving a respiratory airway to reduce hypopnea. One end of the stent body is inserted into the respiratory airway through the human mouth or nasal cavity while the hook at the other end is kept outside the nasal cavity. The end of the stent body entering the respiratory airway is operated to expand, allowing the stent body to keep the genioglossus expand, thereby preventing it from obstructing the airway to cause respiratory pauses. This effectively alleviates the suffocation phenomenon in patients with hypopnea symptom during supine sleep. In addition, the dilating stent is small, and easy and safe to operate with significant use effects, and compared with surgical treatment, almost causes no trauma, meeting medical and home use requirements, thus having tremendous promotional value.

### BRIEF DESCRIPTION OF DRAWINGS

FIG. 1 is a schematic diagram of a stent body being in an expanded state according to Embodiment 1.

FIG. 2 is a schematic diagram of the overall expanded state in Embodiment 1.

FIG. 3 is a schematic structural diagram of a support arm being expanded according to Embodiment 1.

FIG. 4 is a schematic diagram of an overall retracted state according to Embodiment 1.

FIG. 5 is a schematic structural diagram of the support arm being closed according to Embodiment 1.

FIG. 6 is a schematic diagram of an end portion of a sleeve tube according to Embodiment 1.

FIG. 7 is a schematic diagram of an expanded state according to Embodiment 2.

FIG. 8 is a schematic diagram of a retracted state according to Embodiment 2.

FIG. 9 is a schematic diagram of a use state of the present invention.

Numeral references are as follows: 1. sleeve tube, 2. central tube, 3. stent body, 4. support arm, 41. support rod, 42. connection rod, 5. hinge position, 6. hinge shaft, 7. elastic limit block, 8. limit hole, and 9. hook.

### DETAILED DESCRIPTION OF EMBODIMENTS

The accompanying drawings are provided for illustrative purposes only and should not be construed as limitation on the scope of this application. To better illustrate the embodiments, some components in the drawings may be omitted, enlarged, or reduced, which does not necessarily represent the actual product dimensions. It is understandable for those skilled in the art that some known structures and their descriptions in the drawings may be omitted. The positional relationships described in the drawings are provided for illustrative purposes only and should not be construed as limiting the scope of this application.

### Embodiment 1

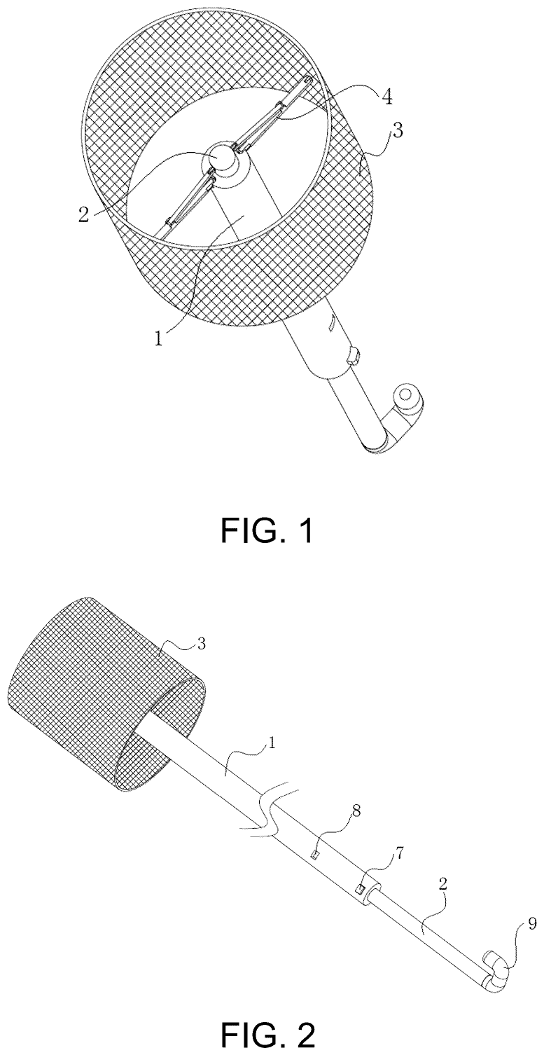

As shown in FIG. 1, this embodiment provides a genioglossus-dilating stent for improving a respiratory airway to reduce hypopnea, mainly used for patients with obstructive sleep apnea syndrome during sleep. In medical diagnosis, a decrease by 15% in normal respiratory ventilation amount is generally considered to be in the severe range of the apnea syndrome. In general, a surgical procedure is usually used for treatment. However, complications from the surgical procedure are risky to some extent, making it unsuitable for all patients. However, the dilating stent, an external tool for auxiliary treatment and obstruction prevention, is free from surgeries, and thus is basically suitable for all patients.

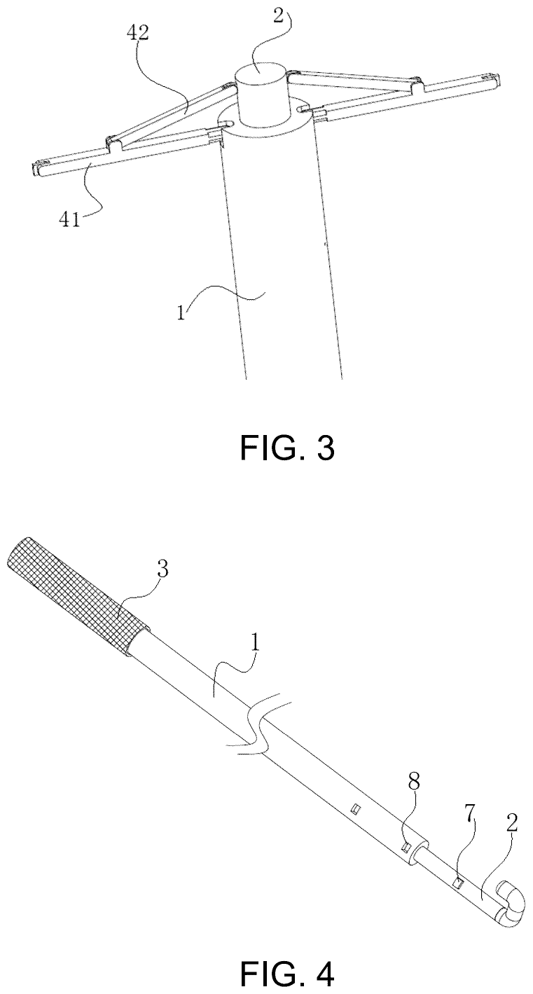

Specifically, as shown in FIGS. 2 to 5, this embodiment includes a sleeve tube 1, a central tube 2, a stent body 3, and multiple support arms 4. The sleeve tube 1 and the central tube 2 are made of a medical-grade semi-flexible material, with requirements for good guiding properties, non-toxicity, and harmlessness. However, the sleeve tube 1 and the central tube 2 need to remain elastic for expansion operation.

The sleeve tube 1 is hollow, and the central tube 2 penetrates through and is sleeved within the sleeve tube 1. An end of the central tube 2 extends from the sleeve tube 1, and the stent body 3 is connected to the end via the support arms 4. During use, the end is inserted from the human nasal cavity or mouth to the position of the genioglossus in the respiratory airway. In addition, one end of the central tube 1 away from the stent body 3 is provided with the hook 9. During use, the hook 9 is hooked on the nose, to prevent the central tube 2 and the sleeve tube 1 from extending too far into the nasal cavity.

Specifically, during retraction, the entire dilating stent is in a shape of a slender and flexible tube, so as to extend into the airway through the nasal cavity or mouth. During use, one end of the stent body 3 is inserted from the human nasal cavity or mouth to the position of the genioglossus in the airway. In this case, the user should maintain a standing or forward-leaning posture to facilitate placement in the position of genioglossus. Subsequently, the stent body 3 is operated to expand, thus preventing the genioglossus from sagging to obstruct breathing when the patient is lying flat.

In this embodiment, the stent body 3 is of a toroidal elastic mesh structure. For the material and specific performance of the stent body 3, reference may be made to heart stents inserted into vessels for support, and the dimension specifications of the stent body 3 are tailored to the dimensions support the respiratory airway. Specifically, the maximum outer diameter of expansion of the stent body 3 is 20 mm. On the basis of the measurement requirements of ventilation amount, it is proper to maintain the width of the respiratory airway in the range of 15 mm to 20 mm. Excessively large expansion is likely to damage the pharyngeal mucosa and capillaries. The pressure after expansion of the stent body 3 does not exceed a 30 mm water column, providing support and avoiding severe compression on the airway mucosa. Slight compression on the mucosa can be maintained for more than eight hours without causing ischemic damage to the mucosa. Furthermore, the height of the stent body 3 in this embodiment is 40 mm, ensuring a sufficient contact area with the genioglossus during expansion. It can be maintained in an elliptical shape under compression for support.

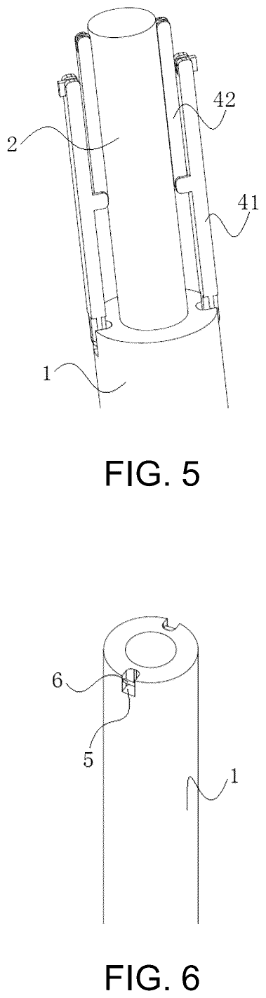

In addition, the toroidal stent body 3 is co-axially arranged with the sleeve tube. As shown in FIGS. 4 and 5, two support arms 4 are provided between the sleeve tube 1 and the stent body 3, and the support arm is connected to the inner wall of the stent body. The support arms 4 are symmetrically arranged around a central axis of the sleeve tube 1, and the two support arms 4 have an included angle of 180°. In addition, the support arm 4 is of a foldable structure, and the support arm 4 has one end connected to end portions of the sleeve tube 1 and the central tube 2 and the other end connected to the inner wall of the stent body 3. During retraction, the support arms 4 are folded, and the stent body 3 is retracted at the end portion of the sleeve tube 1; and during expansion, the support arms 4 open to expand the stent body 3.

Specifically, the support arm 4 includes a support rod 41 and a connection rod 42, and the support rod 41 has one end hinged to the sleeve tube 1 and the other end hinged to the inner wall of the stent body 3. The support rod 41 drags the stent body 3 to expand or retract. The central tube 2 penetrates through the sleeve tube 1, and therefore, the connection rod 42 has one end hinged to the end portion extending from the central tube 2 and the other end connected to the middle part of the support rod 41, such that the connection rod 42 is in linkage with the support rod 41.

In addition, the end portion of the sleeve tube 1 is provided with a hinge position 5 where the support arm 4 is hinged. A hinge shaft 6 is provided in the hinge position 5, and the end portion of the support rod 41 is connected to the hinge shaft 6 and accommodated in the hinge position 5. As shown in FIG. 6, during expansion, the support rod 41 is in a horizontal position or close to the horizontal position. During retraction, the support rod is folded and in or close to the vertical position. Therefore, the movement range of the support rod 41 is 0° to 90°.

In addition, the central tube 2 is longer than the sleeve tube 1, and the two are in clearance fit. One end of the central tube 2 away from the stent body 3 is provided with an elastic limit block 7, and the sleeve tube 1 in this embodiment is provided with two limit holes 8 in fit with the elastic limit blocks. During expansion, the elastic limit block 7 is stuck in one of the limit holes 8, keeping the central tube 2 relatively fixed to the sleeve tube 1. The two limit holes 8 in different sizes correspond to different expansion sizes of the stent bodies 3. Based on requirements, a limit hole 8 corresponding to a large expansion size may be selected to be in the largest ventilation size; or a limit hole 8 corresponding to a moderate expansion size may be selected to be in a size with small support force.

During use, one end of the stent body 3 is inserted into the human respiratory airway, while the other end with the hook 9 remains outside the body. The central tube 2 and the sleeve tube 1 outside the body are operated. Specifically, one end of the central tube 2 is gripped to keep it fixed, and then the sleeve tube 1 is moved to push the support arm 4 at the end of the sleeve tube 1 to open, thus expanding the stent body 3. After expansion through support, to prevent the stent body 3 from being retracted due to the pressure of the throat muscles, it is necessary to maintain the support force of the support arms 4 on the stent body 3. Therefore, it is essential to keep the relative position between the central tube 2 and sleeve tube 1 fixed without undesired sliding. Therefore, the central tube 2 and the sleeve tube 1 are effectively fixed with the elastic limit blocks 7, as shown in FIG. 6.

The dilating stent is typically used under medical advice, specifically for patients with obstructive sleep apnea syndrome, to prevent the genioglossus from sagging and obstructing the airway during supine sleep of the patient to cause respiratory pauses.

When placing the dilating stent, the user should first maintain a standing or forward-leaning posture. At this moment, the entire dilating stent is in a retracted state and in a shape of a slender and flexible tube. One end of the stent body 3 is inserted from the human nasal cavity or mouth to the position of the genioglossus in the airway, and the other end remains outside the body. Then the central tube 2 and the sleeve tube 1 outside the body are operated to expand the stent body 3, as shown in FIG. 9.

The specific operation is as follows: One hand grips one end of the central tube 2 outside the body and keeps it fixed, and then the other hand pushes the sleeve tube 1, allowing the sleeve tube 1 to move to the end at which the stent body 3 is located. In this case, the support arm 4 in the airway is subjected to the push force of the sleeve tube 1. As the hinged points between the ends of the support rod 41 as well as the connection rod 42 and the sleeve tube 1, the central tube 2, and the inner wall of the stent body 3 are correspondingly subjected to forces, resulting in rotation and expansion, the support arms 4 in a folded state slowly expand outward toward the periphery, allowing for the slow expansion of the stent body 3, forming a circular shape of a large outer diameter, thus providing support.

As the sleeve tube 1 slides along the central tube 2, when the limit hole 8 of the central tube 1 reaches the position where the elastic limit block 7 of the central tube 2 is located, the elastic limit block 7 is stuck in the limit hole 8, and the central tube 2 remains relatively fixed to the sleeve tube 1. In this case, the support arms 4 are also in the best expanded state, and the stent body 3 provides stable support to expand the genioglossus, thereby preventing it from obstructing the airway to cause respiratory pauses. At last, the hook 9 is hooked on the nose, to prevent the central tube 2 and the sleeve tube 1 from extending too far into the nasal cavity.

When the dilating stent is retracted, the elastic limit block 7 only needs to be compressed for separation from the limit hole 8. The sleeve tube 1 is then pulled in the opposite direction to close the support arms 4, so as to drive the stent body 3 to be retracted. After the entire stent body 3 is retracted to be in the shape of a slender and soft tube, it is taken out of the airway, thus completing the while operation.

The dilating stent is small, and easy and safe to operate with significant ventilation effects during use, and compared with surgical treatment, almost causes no trauma, meeting medical and home use requirements, thus having tremendous promotional value.

### Embodiment 2

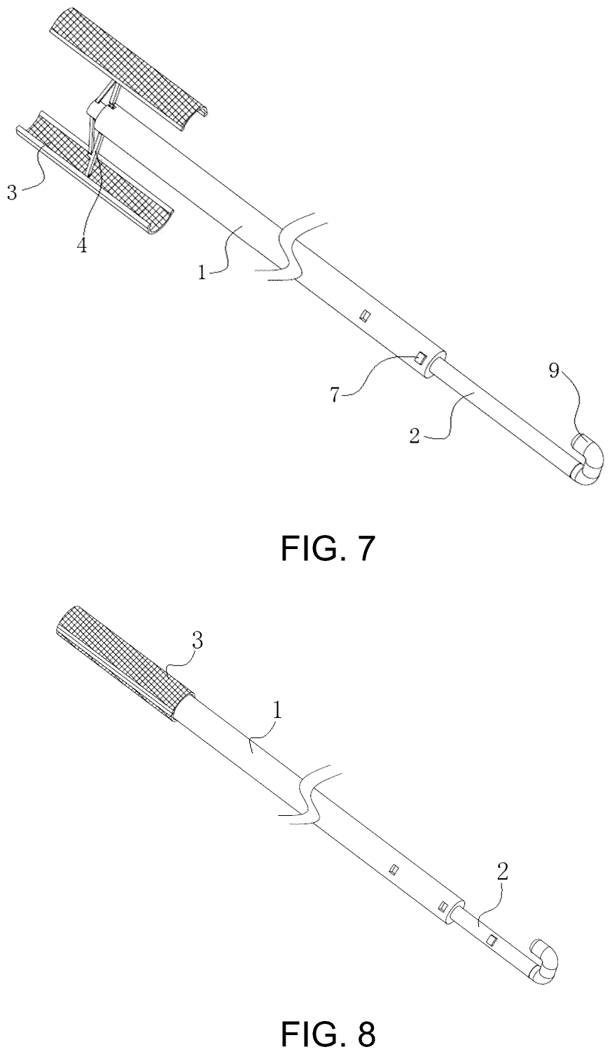

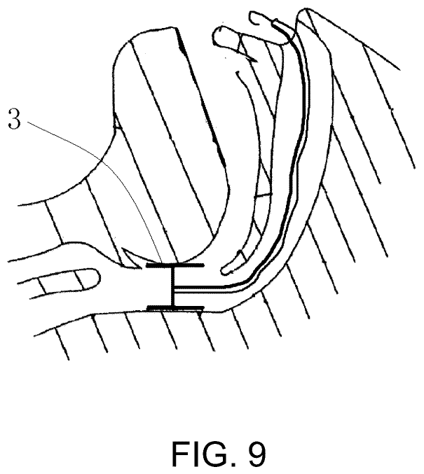

As shown in FIGS. 7 and 8, this embodiment provides another genioglossus-dilating stent for improving a respiratory airway to reduce hypopnea, having the use method and principle similar to those of Embodiment 1, with the difference that the stent body 3 in this embodiment is an arc-shaped structure with a central angle not greater than 180°, where the stent body may be an elastic mesh structure or a thin-sheet structure.

The stent body 3 and the support arm 4 are provided in the same quantity. Two stent bodies 3 and two support arms 4 are provided in this embodiment, and the support arms 4 are connected to the stent bodies 3 in one-to-one correspondence. The support arm 4 has one end connected to the end portions of the sleeve tube 1 and the central tube 2 and the other end connected to the inner wall of the stent body 3, and the two support arms are arranged symmetrically. Two ends of the stent body 3 in the arc-shaped direction are rounded to prevent sharp corners from injuring the wall of the respiratory airway.

Specifically, the stent bodies 3 are two independent arc-shaped structures. The two arc-shaped stent bodies 3 form a circular shape when retracted and combined. The entire dilating stent is still in the shape of a slender and soft tube, so as to be inserted into the airway through the nasal cavity. The maximum outer diameters of expansions of the stent bodies 3 are both controlled to be 20 mm, and the height of the stent body 3 is 30 mm.

Specifically, one support arm 4 is correspondingly connected to an arc-shaped stent body 3 and the support arm 4 is connected to the inner wall of the stent body 3. When the two arc-shaped stent bodies 3 expand, the support arms 4 push the stent bodies 3 to expand along the diameter towards straight lines at two ends, thus providing support. As shown in FIG. 9, the stent body 3 is relatively small with respect to the entire circular shape, and therefore when it expands in the airway, the expansion direction of the stent body 3 needs to face the direction of the genioglossus. Only the airway is expanded in the front and back directions, the expansion effect can be achieved. This process needs to be determined by professional physicians or the patients based on their sensations during the operation.

Clearly, the above embodiments of the present invention are provided solely for illustrating the examples of the present invention and should not be considered as limitation on the implementation of the present invention. For ordinary skilled persons in this field, additional changes or modifications in different forms can be made based on the above description. It is neither necessary nor feasible to exhaustively enumerate all possible embodiments herein. Any modification, equivalent replacement, improvement, or the like made within the spirit and principle of the present invention shall fall within the protection scope of the claims of the present invention.

1. A dilation stent for genioglossus muscle for improving respiratory airway during hypopnea, applied to support genioglossus, and comprising: a sleeve tube, a central tube, and stent bodies, wherein the central tube penetrates through and is sleeved within the sleeve tube, and the stent bodies are connected to an end portion of the sleeve tube;
a plurality of support arms are provided between the sleeve tube and the stent bodies, the support arm is of a foldable structure, and the support arm has one end connected to the sleeve tube and the central tube and the other end connected to the stent body; the central tube is longer than the sleeve tube, and the central tube and the sleeve tube are in clearance fit; and one end of the central tube away from the stent bodies is provided with a hook;
wherein the support arms are provided in a quantity of two, and the two support arms are symmetrically arranged around a central axis of the sleeve tube, the stent body is of an arc-shaped structure with a central angle not greater than 180°, the stent bodies and the support arms are provided in a same quantity, and during retraction, the stent bodies gather to be in a toroidal shape; and the support arms are connected to the stent bodies in one-to-one correspondence,
during retraction, the support arms are folded, and the stent bodies are retracted at the end portion of the sleeve tube; and
during expansion, the sleeve tube pushes the support arms to open, so as to expand the stent bodies.

2. (canceled)

3. (canceled)

4. (canceled)

5. (canceled)

6. The dilation stent for genioglossus muscle for improving respiratory airway during hypopnea according to claim 1, wherein an outer diameter of expansion of the stent body is 15 mm to 25 mm.

7. The dilation stent for genioglossus muscle for improving respiratory airway during hypopnea according to claim 6, wherein the support arm comprises a support rod and a connection rod, and the support rod has one end hinged to the sleeve tube and the other end hinged to an inner wall of the stent body; and the connection rod has one end hinged to an end portion of the central tube and the other end hinged to a middle part of the support rod.

8. The dilation stent for genioglossus muscle for improving respiratory airway during hypopnea according to claim 7, wherein the sleeve tube and the central tube are both made of a bendable and flexible material.

9. The dilation stent for genioglossus muscle for improving respiratory airway during hypopnea according to claim 8, wherein one end of the central tube away from the stent body is provided with an elastic limit block, the sleeve tube is provided with a plurality of limit holes in fit with the elastic limit block, and the limit holes are spaced apart linearly and uniformly; and
during expansion, the elastic limit block is stuck in one of the limit holes, keeping the central tube relatively fixed to the sleeve tube.
